SEQUENCE LISTING (1 sequence)

SEQ ID NO: 1 - DNA, 461 bp, artificial sequence
  non-coding region of the isolated cDNA clone (5-1-1) from hepatitis C virus
atttaggtgacactatagaatactcaagctatgcatcaagcttggtaccgagctcggatccactagtaacggaagaaagtgtgctggaattcgcccttcactccaccatgaatcactcccatgtgaggaactactgtcttcacgcagaaagcgtctagccatggcgttagtatgagtgtcgtacagcctccaggaccccccctcccgggagagccatagtggtctgcggaaccggtgagtacaccggaatcgccaggacgaccgggtcctttcttggataaacccgctcaatgcctggaaatttgggcgtgccccagcaagactgctagccgagtagtgttgggtcgcgaaaggccttgtggtactgcctgatagggtgcttgcgagtgccccgggaggtctcgtagaccgtgcaccagggcgaattctgcagatatccatcacactggcggccgctcgag

(19) United States
(12) Patent Application Publication
(10) Pub. No.: US 2010/0323344 A1
(43) Pub. Date: Dec. 23, 2010

(54) Detection of hepatitis C virus RNA

(76) Inventors: Monique R. Ferguson, Dickinson, TX (US); Guangyu Li, Galveston, TX (US)

Correspondence Address: Benjamin Aaron Adler;ADLER & ASSOCIATES, 8011 Candle Lane, Houston, TX, 77071, US

(21) Appl. No.: 12/802,981

(22) Filed: Jun. 17, 2010

Related U.S. Application Data
(60) Provisional application No. 61/268,960, filed on Jun. 18, 2009.

(51) Int. Cl. C12Q 1/70

(52) U.S. Cl. 435/5

(57) ABSTRACT

The methods and compositions of this invention provide a means for determining the presence of Hepatitis C virus (HCV) in a sample from an individual. This is accomplished by hybridization to tissue samples using a cRNA probe that is specific for HCV. These means allow superior detection of HCV infection by virtue of the RNA riboprobe.

### CROSS-REFERENCE TO RELATED APPLICATIONS

This non-provisional patent application claims benefit of priority under 35 U.S.C. §119(e) of provisional U.S. Ser. No. 61/268,960, filed Jun. 18, 2009, now abandoned; contents of which are incorporated in entirety herein.

### BACKGROUND OF THE INVENTION

1. Field of the Invention

The present invention generally relates to the field of viral detection. More particularly, the present invention relates to novel compositions and methods of detecting Hepatitis C virus.

2. Description of the Related Art

Hepatitis C Virus (HCV) accounts for nearly all cases of non-A, non-B hepatitis (NANBH) and is a persistent health threat worldwide, with more than one million new cases reported annually. HCV infection is almost always chronic and persistent. The most severe consequences of HCV infection are chronic liver disease and death, and HCV infection is the primary impetus for liver transplantation in the US.

Hepatitis C virus (HCV), a member of the Flaviviridae virus family, is an important cause of chronic liver disease leading to cirrhosis and end-stage liver disease in humans. Chronic hepatitis C is now recognized as a leading indication for orthotopic liver transplantation in the United States. HCV is a positive, single-stranded RNA virus. The genome is approximately 10,000 nucleotides and encodes a single polyprotein of about 3,000 amino acids.

The polyprotein is processed by a host cell and viral proteases into three major structural proteins and several non-structural proteins necessary for viral replication. Several different genotypes of HCV with slightly different genomic sequences have been identified that correlate with differences in response to treatment with interferon alpha. Although the assessment of HCV by molecular biological methods, which include ELISA, PCR, sequencing of the entire genome, and propagating HCV in a permissive cell culture system, has been established, detection of HCV genome (positive-strain RNA) and replicative-intermediate RNA (negative-strain RNA) still exists as a problem with current detection methods.

HCV presumably replicates via a negative strand RNA intermediate. Active replication in infected cells is demonstrated by the detection of anti-genomic strand RNA molecules. Detection of the hepatitis C virus (HCV) genome in clinical specimens is useful for differential diagnosis, particularly between recurrent HCV infection, and cellular rejection.

In situ hybridization (ISH) enables visualization of specific nucleic acid in morphologically preserved cells and tissue sections. Several reports have described the detection of HCV RNA in several types of cells or liver tissue by the techniques of in situ hybridization. However, several issues remain unresolved due to conflicting data.

In some studies, only a subset of biopsies from patients with hepatitis C stained positive for HCV RNA, and only a small percentage of hepatocytes appeared to be infected. In other studies, HCV genomes were found in over 90% of biopsies from infected patients. In addition, some reports suggested no relationship between the level of HCV RNA in the liver and the degree of hepatocellular injury; however, other reports have suggested a correlation between HCV RNA in the liver and the degree of liver injury.

There is a recognized need in the art for the development and optimization of a quantitative and highly sensitive strand-specific in situ hybridization assay for detecting HCV RNAs in liver tissue. The present invention fulfills this longstanding need and desire in the art.

### SUMMARY OF THE INVENTION

In one embodiment, the present invention provides a method of detecting Hepatitis C virus (HCV) in tissue samples from individuals, comprising the steps of: contacting the sample with a cRNA riboprobe; hybridizing the cRNA riboprobe to HCV RNA in the tissue samples under conditions wherein the riboprobe hybridizes specifically to HCV nucleic acid; and detecting the hybridized riboprobe as an indication of the presence of HCV in the samples.

In certain embodiments of the above, the hybridization is in-situ hybridization. In certain embodiments, the cRNA riboprobe comprises the nucleotide sequence comprising at least 100 contiguous nucleotides of the sequence of SEQ ID NO: 1; or the nucleotide sequence that is fully complementary to at least 100 contiguous nucleotides of the sequence of SEQ ID NO: 1; or a combination thereof. In these embodiments, the tissue samples are formalin-fixed, paraffin-embedded tissue samples.

In another embodiment of the instant invention is a kit for detecting the presence of HCV RNA in a biological sample wherein said biological sample is a formalin-fixed, paraffin-embedded tissue sample. The kit comprises a cRNA riboprobe comprising a nucleotide sequence comprising at least 100 contiguous nucleotides of the sequence of SEQ ID NO: 1 or the complement thereof; apparatus for performing in-situ hybridization on said biological sample with said riboprobe under conditions wherein said riboprobe hybridizes specifically to HCV nucleic acid; and means of detecting the hybridization between the sample and the riboprobe.

In certain embodiments of the kit described above, the means for detecting the hybridization is by providing riboprobe with a label attached. In some of these embodiments, the label is digoxigenin.

In one embodiment, the present invention provides a composition useful for detection of Hepatitis C virus (HCV) in tissue samples from individuals, said composition comprising of: a digoxigenin labeled cRNA riboprobe comprising a nucleotide sequence comprising at least 100 contiguous nucleotides of the sequence of SEQ ID NO: 1 or the complement thereof.

Other and further aspects, features, and advantages of the present invention will be apparent from the following description of the previously preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows an in situ analysis of HCV replicon RNAs in cell lines with Dig-labeled riboprobes. The Huh-7 cell line with HCV replicon (which express positive and negative strand HCV RNA) and control Huh-7 cell lines were used to analyze efficiency of Dig-labeled riboprobes. The control probe was used as a positive control.

FIG. 2A-2C show in situ analysis of HCV RNAs in frozen liver tissue sections with Dig-labeled riboprobes. FIG. 2A shows detection of the HCV genomic strand by antisense RNA probe. ISH reaction product appears as dark staining within hepatocyte cytoplasm with peri-nuclear intensification. FIG. 2B shows detection of the HCV replicative-intermediate RNA by sense RNA probe. FIG. 2C shows detection of the HCV genomic strand by antisense RNA probe (known as positive). A similar pattern of staining was seen as with the antisense RNA probe.

FIG. 3 shows blind studies using 7 formalin-fixed, paraffin-embedded liver tissues (patients with and without HCV infection). The data is consistent with the actual clinical diagnostic information revealed.

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Subject” means a human or non-human animal selected for treatment or therapy.

“Subject suspected of having” means a subject exhibiting one or more clinical indicators of a disease or condition.

The present study describes the development and optimization of a new quantitative and highly sensitive strand-specific ISH assay for detecting HCV RNAs in liver tissue. With this assay, different and distinct intrahepatic distribution patterns can be observed for HCV genomic and replicative-intermediate RNAs, and also for the correlation between hepatic inflammation and histopathologic assessment of liver diseases.

Hepatitis C virus (HCV) is one of the most important causes of chronic liver disease in the United States accounting for about 15 percent of acute viral hepatitis, 60 to 70 percent of chronic hepatitis, and up to 50 percent of cirrhosis, end-stage liver disease, and liver cancer. Of the United States population, 1.6 percent, or an estimated 4.1 million Americans, have antibody to HCV, indicating ongoing or previous infection with the virus. Hepatitis C causes an estimated 10,000 to 12,000 deaths annually in the United States, and about half of cases of primary liver cancer in the developed world.

The present invention provides compositions and methods to detect the hepatitis C virus (HCV) genome and antigenomes in clinical specimens, which can be used for differential diagnosis, particularly between recurrent HCV infection and cellular rejection. With this assay, different and distinct intrahepatic distribution patterns can be observed for HCV genomic and replicative-intermediate RNAs, and can also be used for dissection of the correlation between hepatic inflammation and histopathologic assessment of liver diseases.

Hepatitis C is most readily diagnosed when serum aminotransferases are elevated and anti-HCV (by method of EIA) is present in serum. The diagnosis is confirmed by the finding of HCV RNA (PCR method) in serum. However, these methods do not provide any information on grading the severity of disease, and staging the degree of fibrosis and permanent architectural damage. Liver biopsy is considered the “gold standard” for assessing the severity of liver disease, and is also helpful in ruling out other causes of liver disease, such as alcoholic liver injury, nonalcoholic fatty liver disease, or iron overload.

In addition, by assigning scores of severity, grading and staging of hepatitis are helpful in managing patients with chronic hepatitis, and particularly helpful in clinical studies on chronic hepatitis. Furthermore, monitoring HCV RNA levels during the early phases of treatment may provide early information on the likelihood of a response. However, because of the absence of an assay for HCV RNA level in liver biopsy, no test is a reliable guide to therapy. Hematoxylin and eosin stains and Masson's trichrome stain are used to grade the amount of necrosis and inflammation and to stage the degree of fibrosis. Specific immunohistochemical stains for HCV have not been developed for routine use.

The present invention provides for the development and optimization of an in situ hybridization (ISH) assay to demonstrate HCV infection in liver biopsies from patients with HCV infection. Specifically, more sensitive and specific probes for identifying HCV genome and antigenome specific regions were obtained. The specific cRNA probes, along with the optimized in situ hybridization assay system, can be developed into diagnostic kit for promoting public health, and also have potential benefits to further biomedical research capabilities.

Two labeled complementary RNA probes with optimized in-situ hybridization assay as described herein can be used to localize HCV RNA sequences in section of tissue (in-situ). RNA in-situ hybridization is used to measure and localize mRNAs and other transcripts within tissue sections or whole amounts of tissue. Sample cells and tissues are usually treated to fix the target HCV transcripts in order to increase access to the probes. These probes are two complementary RNAs (riboprobes). Riboprobes are more sensitive than traditional oligoprobes and actually better than a cocktail of oligoprobes. An oligoprobe usually incorporates 1˜2 labeling molecules for each oligoprobe at one or both ends, while a riboprobe can incorporate 20 to 100 labeling molecules. During in situ hybridization, two probes hybridize to the target sequences at elevated temperature, and then the excess probes are washed away (after prior hydrolysis using RNase in the case of un-hybridized, excess RNA probe). Solution parameters such as temperature, salt and/or detergent concentration can be manipulated to remove any non-identical interactions (i.e. only exact sequence matches will remain bound). Then, the digoxigenin-labeled probes are localized and quantitated in the tissue using immunohistochemistry. These operating procedures provide efficiently optimized staining conditions for these two novel probes.

### Example 1

### Cell Lines and HCV Replicons

Huh-7 cell line and Huh-7 cells containing HCV replicons for optimizing the ISH assay were obtained from laboratory of Dr. Stanley Lemon (Director, Institute for Human Infections and Immunity, University of Texas Medical Branch, Galveston, Tex.). Huh-7 cell line, which are human hepatocellular carcinoma cells, were cultured in Dulbecco's modified Eagle medium (DMEM) containing high levels of glucose (0.45%) and supplemented with 10% fetal bovine serum, 50 U of penicillin per ml, and 50 ug of streptomycin per ml at 37° C. in 5% CO₂. Huh-7 cell with HCV replicons expressing either HCV positive-strand (genomic) or HCV replicative-intermediate (antigenomic) RNA was cultured in same culture system above in the presence of 10 ug of blastacindin (Roche Biochemicals, Indianapolis, Ind.).

To prepare the cell lines for in situ hybridization analysis, Huh-7 cells and Huh-7 cells with HCV replicons were grown in 8-well chamber slides (1×10⁵ cells/well), respectively, supplemented with growth medium. Thirty-six hours later, the supernatant was discarded, and cells were washed with PBS. Then cells were fixed in 10% formalin (pH 7.4) in a coplin jar for 10 min at room temperature, washed twice with PBS, dried and stored at −80° C. until in situ hybridization analysis.

### Example 2

### Collection and Processing the Liver Tissues

Liver needle biopsy specimens were obtained from patients at John Sealy Hospital of University of Texas Medical Branch, Galveston, Tex. under informed consent. Immediately after being released from the needle, the core biopsies were processed by formalin fixation and paraffin embedding for histological examination and HCV RNA testing by in situ hybridization. Frozen liver sections (6 μm) with HCV infection prepared for ISH assay were initially placed on 50° C. heating blocks for 5 min to improve adherence, fixed in 10% formalin for 5 minutes at room temperature, and sequentially washed twice in PBS 10 minutes each, dried and stored at −80° C. for later use hepatocellular carcinoma HCC liver sections were presented as positive control by Dr. Lemon. Formalin-fixed paraffin-embedded liver tissues (patients with hepatitis C or without HCV infection) were provided by Dr. Pfeiffer of the Washington University School of Medicine.

### Example 3

### Preparation of cDNA Clone and Riboprobes

The pCRII-TOPO-HCV vector that contains the 319 bp of cDNA corresponding to the 5′ non-coding region of HCV genome was also presented by Dr. Pfeiffer. The plasmid DNAs for both forward and reverse orientation were linearized with Xho I and BamH I respectively. Sense RNA transcripts were generated by Sp6 RNA polymerase using the transcription in Vitro System kit with digoxigenin-11-UTP (Roche Biochemicals, Indianapolis, Ind.), and antisense transcripts were generated by T7 RNA polymerase using same transcription kit according to the manufacture's protocol. The production of RNA and the subsequent removal of the cDNA template were monitored by denaturing gel electrophoresis and then visualized about 454 bp for sense probes and 445 bp for antisense probes. Dig-labeled riboprobes were further broken down to an average size of 100 nucleotides by alkaline hydrolysis. A riboprobe specific for HCV genome was used for positive control (provided by Dr. Lemon from University of Texas Medical Branch, Galveston, Tex.).

### Example 4

### In Situ Hybridization

Paraffin embedded slides were dipped in xylene for 10 min, then in other pure xylene for 10 min to remove the wax, and rehydrated in 100% ethanol, 100% ethanol, 95% ethanol, 80% ethanol and pure H₂O 5 min each. Frozen sections and cell cultured slides were taken out from −80° C. freezer. All slides were treated in 2×SSC for 15 min and place in proteinase K in TE solution (1 ug/ml) at 37° C. for 30 min, and stopped in glycine for 2 min. Slides were washed in PBS twice at 5 min each and post-fixed in 4% PFA for 20 min, then incubated slides twice in PBS, 15 min each, put slides into 5×SSC (in fume hood). Meanwhile, prepared the pre-hybridization solution (Per 1 ml: 499 ul H₂O, 250 ul 20×SSC, 500 ul formamide and 10.6 mg salmon sperm DNA), heat it to 80° C. for 10 min and cool on ice. Each slide was taken out, carefully added 200 ul of pre-hybridization solution, covered with a small strip of parafilm, placed in a humidity chamber, and incubated the chamber at 58° C. for 2 hours. Prepared probes 30 ng per slides, and denatured the probes by heating to 80° C. for 5 min and cooling on ice. Carefully drained off the pre-hybridization solution and pipette a minimal amount of probe in hybridization solution (100 ul) on each slide and replaced the parafilm, and incubated at 60° C. in a humidified chamber for 36 hours. After hybridization, the slides were put back in racks and into 2×SSC for 1 hour, then changed slides into 0.1×SSC at 65° C. for 2 hours.

### Example 5

### Immunological Detection

Tissue sections were gently immersed in buffer 1 (100 mM Tris-HCl pH7.5 and 150 mM NaCl) at 40° C. and cooled to room temperature for 15 minutes, followed by incubation with blocking solution (100 mM Tris-HCl pH 7.5, 150 mM NaCl and 1% blocking reagent, Roche, Cat. NO. 11096176001) for 45 minutes. The antibody buffer was put (dilution anti-Dig-alkaline phosphatase conjugate in blocking solution) to the sections directly and incubated for 2 h at RT, then wash slides twice in washing buffer for 15 minutes. Sections were equilibrated 5 minutes in buffer 2 (100 mM Tris-HCl pH9.5, and 100 mM NaCl and 50 mM MgCl₂), and covered with 100 ul freshly prepared color substrate solution (dilution of NBT/BCIP by buffer 2 as 1:50) in an appropriate container in the dark for 16 h. After reaction, slides were rinsed in H₂O and observed under a microscope, then mount in slightly warmed glycerol to preserve the slides.

### Example 6

### Characterization of Dig-Labeled Riboprobes

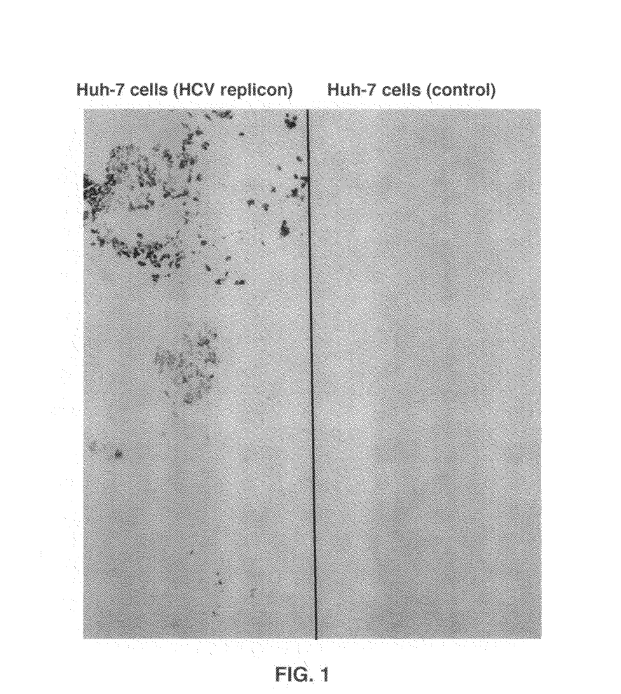

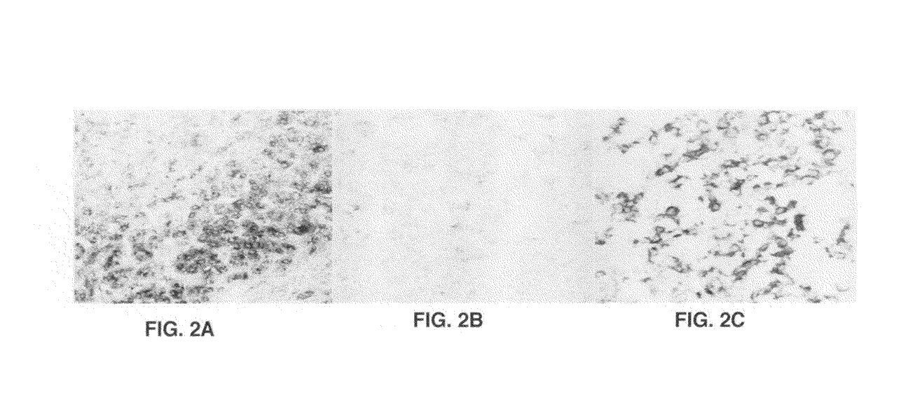

Huh-7 cell line with HCV replicons, Huh-7 cell lines and frozen liver tissue sections were used to analyze efficiency of Dig-labeled riboprobes. Control probe was used as positive control. As FIGS. 1 and 2 illustrates, high sensitivity and specificity of the riboprobes were obtained and optimized.

### Example 7

### In Situ Analysis of HCV Genomic and Anitgenomic RNAs in Human Liver Tissues

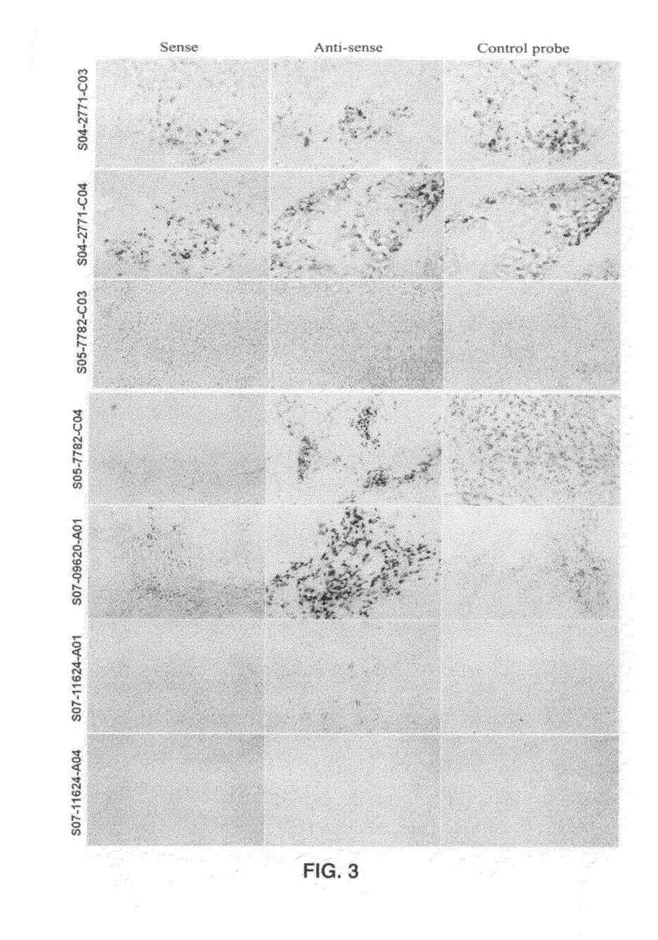

Total seven formalin-fixed, paraffin-embedded liver tissues (patients with hepatitis C or without HCV infection) were detected blindly. As the FIG. 3 shows, four liver tissues were positive, and three negative.

Pathologists can use these developed and optimized cRNA probes in an in situ hybridization assay to detect the HCV genome and replication from in liver biopsies or formalin-fixed, paraffin-embedded tissues from patients who have liver transplant for chronic hepatitis C, and also help differentiating recurrent HCV infection and cellular rejection in difficult cases. The present invention provides an optimized assay which can be developed into a diagnostic kit.

Maxim Biotech, Inc produces a biotin-conjugated cDNA probe for HCV (catalog number IH-60053), and an in-situ hybridization/detection kit (IHD-0050 & IHD-0052). Use of the Maxim Biotech, Inc kits to detect the HCV RNA in biopsy sections were unsuccessful. The cRNA probes disclosed herein are 10- to 100-fold more sensitive than the cDNA probe for HCV RNAs detection. In addition, the probes disclosed herein are very stable because 10 times freeze/thaw cycles don't affect their function. This optimized in situ hybridization assay has been verified in formalin-fixed, paraffin-embedded biopsy tissues from patients with or without HCV infection, bi-blindly.

Prior art methods are unable to grade the severity of disease, and staging the degree of fibrosis and permanent architectural damage. Liver biopsy is considered the “gold standard” for assessing the severity of liver disease, and is also helpful in ruling out other causes of liver disease, such as alcoholic liver injury, non-alcoholic fatty liver disease, or iron overload. By assigning scores of severity, grading and staging of hepatitis are helpful in managing patients with chronic hepatitis, and particularly helpful in clinical studies on chronic hepatitis. Furthermore, monitoring HCV RNA levels during the early phases of treatment may provide early information on the likelihood of a response. Hematoxylin and eosin stains and Masson's trichrome stain are used to grade the amount of necrosis and inflammation and to stage the degree of fibrosis. Specific immunohistochemical stains for HCV have not been developed for routine use. The present invention provides an optimized, in-situ hybridization assay to demonstrate HCV infection in liver biopsies from patients with HCV infection, using sensitive and specific probes for identifying the HCV genome and antigenome specific regions.

In situ hybridization enables visualization of specific nucleic acid residues in morphologically preserved cells and tissue sections. In recent years, several papers [Gastaldi, et al. 1995; Blight, et al. 1992; Lau, et al. 1996; Gosalvez, et al. 1998; Tanaka, et al. 1993; Negro, et al. 1998; Nouri, et al. 1993; Agnello, et al. 1998; Arrieta, et al. 2000; Chang, et al. 2000; Targett-Adams, et al. 2008] have described conflicting data for the detection of HCV RNA in cells and liver tissue by ISH. However, the current invention describes the development and optimization of a quantitative and highly sensitive and specific in situ hybridization assay for detecting HCV RNAs in FFPE liver tissues. The probes herein are digoxigenin-labeled cRNA probes, and more sensitive and specific than their biotin-conjugated cDNA probe. Additionally, these probes are very stable because 10 times freeze/thaw cycles don't affect their function. The specific cRNA probes, along with the optimized in situ hybridization assay system, can be developed into diagnostic kit for promoting public health, and also have potential benefits to further biomedical research capabilities.

### Sense Probe:

SEQ ID NO: 1:
5′atttaggtgacactatagaatactcaagctatgcatcaagcttggtac
cgagctcggatccactagtaacggccgccagtgtgctggaattcgccctt
cactccaccatgaatcactcccctgtgaggaactactgtcttcacgcaga
aagcgtctagccatggcgttagtatgagtgtcgtacagcctccaggaccc
cccctcccgggagagccatagtggtctgcggaaccggtgagtacaccgga
atcgccaggacgaccgggtcctttcttggataaacccgctcaatgcctgg
aaatttgggcgtgcccccgcaagactgctagccgagtagtgttgggtcgc
gaaaggccttgtggtactgcctgatagggtgcttgcgagtgccccgggag
gtctcgtagaccgtgcaccagggcgaattctgcagatatccatcacactg
gcggccgctcgag-3′

### Antisense Probe:

Complemented to sense probe, only difference in 5′ and 3′ terminal, which comes from vectors.

### REFERENCES

Agnello et al., Hepatology. 1998; 28(2):573-584.
Arrieta et al., Hepatology. 2000; 32(1):97-103.
Blight K, Trowbridge R, Rowland R, Gowans E. Liver. 1992; 12:286-289.
Chang et al., J. Virol. 2000; 74(2):944-55.
Choo et al., Science. 1989 Apr. 21; 244(4902):359-62.
Gastaldi et al., J. Hepatol. 1995; 23(5):509-518.
Gosálvez et al., Hepatology. 1998; 27(5):1428-1434.
Haruna et al., J. Hepatol. 1993; 18(1):96-100.
Lau et al., In situ detection of hepatitis C virus—a critical appraisal. J. Hepatol. 1996; 24(2 suppl):43-51.
Nouri et al., J Clin Invest. 1993; 91(5):2226-2234.
Sansonno et al., Pathobiology. 1995; 63(5):239-248.
Tanaka et al., Liver. 1993; 13(4):203-208.
Targett-Adams P, Boulant S, McLauchlan J. J. Virol. 2008; 82(5):2182-2195.

What is claimed is:

1. A method of detecting Hepatitis C virus (HCV) in tissue samples from individuals, comprising the steps of:
contacting said sample with a cRNA riboprobe;
hybridizing said riboprobe to HCV RNA in said samples under conditions wherein said riboprobe hybridizes specifically to HCV nucleic acid; and
detecting said hybridized riboprobe as an indication of the presence of HCV in said samples.

2. The method of claim 1 wherein said hybridization is by in-situ hybridization.

3. The method of claim 1, wherein said cRNA riboprobe comprises nucleotides in the 5′-non-coding region of the isolated cDNA clone (5-1-1) from the genome of an infectious human hepatitis C virus that is immunologically unrelated to the hepatitis A and hepatitis B viruses.

4. The method of claim 3, wherein said cRNA riboprobe comprises one of:
the nucleotide sequence comprising at least 100 contiguous nucleotides of the sequence of SEQ ID NO: 1;
the nucleotide sequence that is fully complementary to at least 100 contiguous nucleotides of the sequence of SEQ ID NO: 1; or
a combination thereof.

5. The method of claim 1 wherein said tissue sample is obtained from the liver of said individual.

6. The method of claim 1 wherein said tissue sample is formalin-fixed, paraffin-embedded tissue sample.

7. The method of claim 1 wherein said detection of said hybridization is by providing said riboprobe with a label attached.

8. The method of claim 7 wherein the label is digoxigenin.

9. A method of detecting Hepatitis C virus (HCV) in a tissue sample from an individual comprising the steps of:
fixing the sample in formalin and paraffin-embedding the formalin fixed tissue sample;
contacting the formalin-fixed paraffin-embedded tissue sample with a digoxigenin labeled cRNA riboprobe;
hybridizing said riboprobe to HCV RNA in the sample under conditions wherein said riboprobe hybridizes specifically to HCV nucleic acid; and
detecting said hybridized riboprobe as an indicator of the presence of HCV in said samples.

10. The method of claim 9, wherein said cRNA riboprobe comprises one of:
the nucleotide sequence comprising at least 100 contiguous nucleotides of the sequence of SEQ ID NO: 1;
the nucleotide sequence that is fully complementary to at least 100 contiguous nucleotides of the sequence of SEQ ID NO: 1; or
a combination thereof.

11. The method of claim 9 wherein said tissue sample is obtained from the liver of said individual.

12. A method of detecting Hepatitis C virus (HCV) in a tissue sample from an individual comprising the steps of:
fixing the sample in formalin and paraffin-embedding the formalin fixed tissue sample;
contacting said formalin-fixed paraffin-embedded sample with a digoxigenin labeled cRNA riboprobe comprising a nucleotide sequence comprising at least 100 contiguous nucleotides of the sequence of SEQ ID NO: 1 or the complement thereof;
hybridizing said riboprobe to HCV RNA in the sample under conditions wherein said riboprobe hybridizes specifically to HCV nucleic acid; and
detecting said hybridized riboprobe as an indication of the presence of HCV in said samples.

13. A kit for detecting the presence of HCV RNA in a formalin-fixed, paraffin-embedded biological tissue sample, said kit comprising:
a cRNA riboprobe comprising a nucleotide sequence comprising at least 100 contiguous nucleotides of the sequence of SEQ ID NO: 1 or the complement thereof; and
means of detecting the hybridization between the sample and the riboprobe.

14. The kit of claim 13 wherein the means for detecting said hybridization is by providing said riboprobe with a label attached.

15. The kit of claim 14 wherein the label is digoxigenin.

16. The kit of claim 13 wherein said hybridization is by in-situ hybridization.

17. The kit of claim 13, further comprising apparatus for performing in-situ hybridization on said biological sample with said riboprobe under conditions wherein said riboprobe hybridizes specifically to HCV nucleic acid.

18. A composition useful for detection of Hepatitis C virus (HCV) in formalin-fixed, paraffin-embedded liver tissue samples from individuals, said composition comprising of:
a digoxigenin labeled cRNA riboprobe comprising a nucleotide sequence comprising at least 100 contiguous nucleotides of the sequence of SEQ ID NO: 1 or the complement thereof.
